(19) United States
(12) Patent Application Publication
(10) Pub. No.: US 2010/0261215 A1
(43) Pub. Date: Oct. 14, 2010

(54) NON-INVASIVE METHOD FOR COLLECTING BIOLOGICAL DATA FOR ESTABLISHING A DIAGNOSIS OF A CUTANEOUS PATHOLOGY

(75) Inventors: Bruno MEHUL, Gourdon (FR); Gilbert Laffet, Biot (FR); Luigi Russo, Nice (FR)

Correspondence Address: BUCHANAN, INGERSOLL & ROONEY PC, POST OFFICE BOX 1404, ALEXANDRIA, VA, 22313-1404, US

(73) Assignee: GALDERMA RESEARCH & DEVELOPMENT, BIOT, FRANCE.

(21) Appl. No.: 12/779,073

(22) Filed: May 13, 2010

Related U.S. Application Data
(63) Continuation of application No. PCT/FR2008/052049, filed on Nov. 14, 2008.

(30) Foreign Application Priority Data
Nov. 14, 2007 (FR) 0759039

(51) Int. Cl. C12Q 1/37; G01N 30/72

(52) U.S. Cl. 435/23; 250/282

(57) ABSTRACT

A non-invasive method for collecting biological data useful for establishing a diagnosis or a prognosis of a particular cutaneous pathology in a patient, includes:
a) obtaining a skin sample containing biological material by applying a layer of adhesive on the skin of such patient, and then removing the adhesive containing such skin sample/biological material, and
b) conducting mass spectrometry and detecting, in the sample, at least one protein whose presence, absence, or variation in amount or concentration relative to a standard value is associated with the presence, the development or the absence of a particular cutaneous pathology; the subject methodology is particularly suitable for establishing a diagnosis or a prognosis of psoriasis.

### CROSS-REFERENCE TO PRIOR APPLICATIONS

This application claims priority under 35 U.S.C. §119 of FR 0759039, filed Nov. 14, 2007, and is a continuation/national phase of PCT/FR 2008/052049, filed Nov. 14, 2008 and designating the United States (published in the French language on Jun. 4, 2009 as WO 2009/068825 A1; the title and abstract were also published in English), each hereby expressly incorporated by reference in its entirety and each assigned to the assignee hereof.

### BACKGROUND OF THE INVENTION

1. Technical Field of the Invention

The present invention relates to the application of mass spectrometry based on utilizing a non-invasive skin sample as an aid in the diagnosis, prognosis or therapeutic after-care of skin diseases, such as psoriasis. This invention relates more particularly to a method of analysis of a sample from the upper layers of the epidermis.

2. Description of Background and/or Related and/or Prior Art

Skin diseases are generally diagnosed by biopsy or by local examination of the cutaneous symptoms, such examination involving visual assessment of the zones displaying inflammation. Quite clearly, a non-invasive method, such as visual examination, is preferable to biopsies in terms of costs, time, as well as comfort for the patient. However, in certain cases it is difficult to distinguish effectively by simple visual examination between certain skin pathologies, particularly certain skin disorders accompanied by hyperkeratinization (presence of abundant scales). Thus, errors in diagnosis can occur in particular when the pathologies in question only present initial symptoms or symptoms that are not well-developed. If there is an error in diagnosis, the patient may have to be subjected to a succession of ineffective treatments, which should certainly be avoided. Moreover, a succession of ineffective treatments may also have the result that the patient abandons all treatment.

Furthermore, in the case of cutaneous pathology and especially psoriasis, different patients may respond differently to one and the same treatment even if the diagnosis is correct. Generally, for cutaneous pathologies, and in particular psoriasis, a minimum period of about two weeks is necessary to determine whether the treatment shows positive effects. Thus, there is a need for an effective, rapid and non-invasive method for determining a patient's response to a particular treatment. Such a method would also be of particular interest during clinical tests, by providing rapid identification of the presence of a response to a given treatment and therefore reduction of costs as well as reduction of patients' involvement in ineffective treatments.

The methods employed to date make it possible to identify the presence, absence or change of certain blood or skin markers, but in actual use it has been found that these methods are not very effective, or are difficult to put into practice. Thus, patent application US2005/0221334A describes a method employing skin samples collected using an adhesive applied to the skin. This method then describes the diagnosis of cutaneous pathologies such as eczema or psoriasis by identifying the messenger RNA of certain markers on the samples collected. The messenger RNA is identified using RT-PCR and a “micro-array” (ordered set of samples). This procedure is long and difficult to put into practice in particular for enabling markers to be identified. Moreover, the presence or absence of proteins (markers) is not necessarily linked to the presence of messenger RNA coding for these proteins but can result from post-translational modifications (glycosylation or deglycosylation, glycation, oxidation, phosphorylation etc.) or may be linked to the subsequent fate of these proteins, such as enzymatic hydrolysis for example. This hydrolysis by the action of endogenous proteases can lead to the appearance of mature forms or to the disappearance of certain markers. These events are particularly marked in the layers of the epidermis. These biochemical changes may indicate a condition (disorder) of the epidermis that can only be observed by detecting and identifying the proteins, notably by proteomic analysis.

Patent application WO2007/96846 describes a method of assessing the skin by measuring the metabolites in skin samples. This method makes it possible to monitor the metabolites of the skin surface, which are the consequences and not the actors of the biochemical reactions that have taken place in the skin. The techniques developed in this application are specific to the analysis of the metabolome and are not suitable for analysis of the proteome. One skilled in the art cannot extrapolate this method of evaluation of the metabolome to the identification of protein markers.

Identification of the protein content and its proteomic analysis make it possible to monitor specific markers of a cutaneous pathology and guide the diagnosis. Moreover, most of the therapeutic targets are of the nature of protein, which intensifies the interest in discovering, identifying and monitoring the proteins on the surface of the skin employing a technique that is specific to the latter. The identification of proteins upstream of the metabolome is therefore fundamental for characterization of the tissue response.

Therefore, need exists for a method for determining, quickly, non-invasively and inexpensively, the presence, the absence or the variation of certain proteomic markers, which cannot be detected by technologies such as RT-PCR, indicating the presence of particular cutaneous pathologies.

Although various methods for identifying markers of cutaneous pathologies exist in the prior art, these methods are either long and expensive or have insufficient sensitivity or else they necessitate an invasive procedure such as taking a blood sample or obtaining a skin biopsy, which is often painful and traumatic for the patient.

### SUMMARY OF THE INVENTION

The present invention overcomes the drawbacks of the prior art by offering a novel method that is reliable, rapid and non-invasive, for collecting biological data that can be employed for establishing a diagnosis or a prognosis of a cutaneous pathology such as psoriasis.

The present invention thus features a non-invasive method for collecting biological data that can be used for establishing a diagnosis or a prognosis of a particular cutaneous pathology in a patient, wherein:

a) a skin sample containing biological material is obtained by applying a layer of adhesive on the skin of said patient, and then removing said adhesive,

b) employing mass spectrometry for detecting, in the skin sample obtained in a), at least one protein whose presence, absence, or variation in amount or concentration relative to a standard value is associated with the presence, the development or the absence of a particular cutaneous pathology.

### DETAILED DESCRIPTION OF BEST MODE AND SPECIFIC/PREFERRED EMBODIMENTS OF THE INVENTION

Mass spectrometry (MS) is already used in the identification of complex mixtures of chemical compounds and of molecules of biological origin. In contrast to the tools used conventionally, such as chips or the ELISA technique, this tool makes it possible to visualize novel markers and novel proteins not yet identified, and moreover by a method possessing better sensitivity.

Briefly, the basic principle of analysis by mass spectrometry is measurement of the mass/electric charge ratio of ionic species that have been created by ionization of the sample to be analyzed and to which electric and magnetic fields are applied. One of the methods of ionization most commonly used in the case of samples of biological origin is electrospray (ES), which generates multiply charged ions and permits the use of inexpensive conventional analyzers such as quadrupoles. The ionization source for the electrospray can be multiple, and in particular is able to offer good sensitivity for a low flow rate. Another method of ionization frequently used is matrix-assisted laser desorption/ionization, known by the name MALDI-TOF, which is a method of ionization that is very suitable for time-of-flight analyzers (MALDI for “Matrix-Assisted Laser Desorption/Ionization” and TOF for “Time of Flight”).

The mass/electric charge ratio is measured in various ways, for example by measuring the standard deviation of the ionized species (“Sector MS”) or by measuring the time of flight (“TOF MS”). There are now various methods of analysis, including for example quadrupolar or quadrupole mass analyzers (QMS), quadrupolar ion trap mass analyzers (QIT), Fourier transform infrared spectrometers (FTICR). These analyzers and their methods of use are well known.

The method called tandem mass spectrometry (MS/MS) is a combination of several analyzers, permitting the selection of a particular ion and then identification of its fragments by successively intercalating stages of mass separation with stages of fragmentation. Thus, a tandem analyzer can isolate a peptide resulting from hydrolysis by trypsin, fragments of proteins or one protein of a complex mixture, and then stabilize it to permit its fragmentation (for example by means of a gas or of by some other known method) and catalogue the fragments thus produced. The use of databases then enables the proteins present in the sample to be identified.

Preferably, the method according to the invention employs the technique of tandem mass spectrometry (MS/MS).

If it is sought to undertake the identification of a complex mixture of proteins, it is advantageous to carry out a fractionation stage first. A first method of fractionation makes it possible to separate proteins from one another by two-dimensional electrophoresis in polyacrylamide gel. A second method consists of using liquid chromatography (LC) to separate the proteins before submitting them to enzymatic hydrolysis (generally with trypsin). The peptides (hydrolysate) are then introduced into the mass spectrometer. These two techniques can be combined advantageously. Liquid chromatography used in combination with a mass spectrometer is different from high-performance liquid chromatography (HPLC) that is more generally used, mainly with respect to the dimensions of the columns used and therefore their flow rates. Thus, the diameter of the columns used is most often 1 mm (in contrast to average diameters of 4.6 mm for HPLC columns). More recently, diameters of 300 μm and even columns of the capillary type with diameters of 75 μm have become common. With these columns, the flows are close to 100 nL/min. These columns are generally used with nanospray ionization sources, which offer better sensitivity. A source that is used in particular according to the invention is a nanospray source such as that marketed under the name Nanospray™ by the company Applied Biosystems, using Picotip emitter™, SilicaTip needles, and coupled to a mass spectrometer of the LC/MS/MS type such as that marketed under the trademark Qstar XL™ T by the company Applied Biosystems.

In particular, the analysis by mass spectrometry used according to the invention comprises a stage of separation by liquid chromatography combined with tandem mass spectrometry (MS/MS).

The spots obtained by two-dimensional electrophoresis generally correspond to a single protein. To identify the latter, the gel containing the spot is cut out and is submitted to enzymatic proteolysis. The enzyme most often used is trypsin.

The proteins isolated are identified using the technique of peptide mapping (“peptide mass fingerprinting”), in which the masses of the peptides obtained are compared with a reference list on databases that lists the masses of the peptides resulting from the digestion of known proteins. If the experimental data correspond to a given list, there is a high probability that the protein is present in the initial sample.

Analysis in MS/MS (tandem) mode is particularly recommended for obtaining information on the peptide sequence and thus for evaluating the relevance of the previous identification of the protein (validation). Tandem analysis consists of isolating and then fragmenting the peptide ions in a collision cell. In this method, the site of fragmentation is particularly localized on the peptide bonds. After fragmentation, the resulting mass spectra are compared by software with the reference information contained in databases for identification of proteins.

Alternatively and preferably, it is also possible to perform the enzymatic proteolysis of the mixture of proteins directly and then separate the peptides obtained by enzymatic digestion or the endogenous peptides by nano-chromatography prior to analysis by tandem mass spectrometry (MS/MS).

Thus, as one example, the peptides can be concentrated beforehand on a pre-column in acid medium (C18, pepMap 100, 5 μm, 100 A, 300 μm i.d.×5 mm), installed in a nano-chromatography chain “LC packing” (from the company Dionex). The peptides are then chromatographed on a C18 column, 3 μm, 75×150 mm, Atlantis™ (made by Waters), the progressive elution of the peptides being performed by means of a gradient (acetonitrile/water) coupled directly to the source of the mass spectrometer. Analysis in SM/SM mode is particularly recommended for the identification of proteins from complex mixtures.

In the sense of the present invention the term “biological marker” denotes a biological molecule associated with the presence or with the absence of a particular pathological state, and is more particularly a protein.

“Standard value” means the average amount or concentration of biological marker present in healthy individuals.

The method according to the invention can have various applications. It can be used not only for establishing a diagnosis or a prognosis, but can also be useful for investigating or observing the development of cutaneous pathologies or the effect of particular treatments as well as for establishing a prognosis of the efficacy of a given treatment.

According to a preferred embodiment of the method according to the invention, this method is used for determining the existence of a positive response to the administration of a particular treatment by identifying a significant change in the presence of a particular marker. Employed in this manner, it provides quicker determination of the efficacy of a particular treatment than by waiting for the development of the symptoms to be observed visually. It also makes it possible to reduce the costs associated with therapeutic studies of new medicinal products.

The skin disease, or cutaneous pathology, to which the method of the invention relates can be any cutaneous pathology. The latter is preferably a disease of the inflammatory and/or autoimmune or allergic type.

According to a particularly preferred embodiment of the invention, the cutaneous pathology is psoriasis and the marker to be identified is advantageously a marker of psoriatic skin lesions.

Psoriasis is a skin disease of poorly understood origin, partly genetic, which affects 2% of the population. The epidermis is renewed too quickly, in only four to six days, instead of the usual three weeks, which causes localized inflammations. The epidermal cells accumulate on the surface of the skin and form a layer of white scales. Although completely harmless, they have the disadvantage of being unsightly.

In its benign form, psoriasis is limited to the scalp, the nails, the knees, the elbows, the feet, the hands and, sometimes, the genital organs. In severe cases, it spreads and can affect the whole body. Psoriasis comprises various symptomatic forms such as plaque psoriasis, guttate psoriasis, pustular psoriasis, inverse psoriasis, erythrodermic psoriasis and psoriatic arthritis. It is, however, possible to control psoriasis, to reduce the extent of the lesions and to improve patients' quality of life with certain treatments.

Preferably, according to the invention, the marker of psoriasis that is detected or measured is selected from the group comprising proteins having biological functions connected with cellular proliferation and differentiation. Notably is stratifin, the protein related to calmodulin-like skin protein 5 (CLSP), the protein binding the fatty acid associated with psoriasis, desmoplakin; psoriasin (S100 A7) and alpha enolase, squamous cell carcinoma antigen 1 and 2 (SCCA1, SCCA2).

According to another preferred embodiment of the invention, the marker of psoriasis that is detected or measured is selected for example from the group comprising markers having biological functions connected with the response to stress and with the immune and inflammatory response. Notably are the heat shock proteins 27 (beta 1), 70.1, and 90, Calgranulin A and B.

According to the method of the invention, the stage of detection of a protein as a biological marker comprises the identification or determination of the presence or absence of at least one protein. This stage can optionally also comprise determination of the quantity or of the concentration, relative or otherwise, of said marker, and/or of the variation of these values relative to a given or standard value as well as the variation of these values as a function of time. Although it is currently considered more advantageous for reasons of simplicity of practical application simply to determine the presence of a particular marker, this invention also features the determination of amounts, relative or otherwise, of particular markers as well as investigation of the variation of the concentration of several markers.

According to the method of the invention, at least one skin sample, for example a single sample, in particular from the top layer of the epidermis (the stratum corneum) is taken from a patient. Preferably the sample is taken at a location showing clinical signs of a cutaneous pathology, such as inflammation. However, it may be advantageous to take a sample from skin apparently unaffected as well, especially for comparative studies and/or for confirming the results of the analysis. When the skin sample is taken from an inflamed area, it is preferable for sampling to take place at the centre of the plaque, so as to optimize sampling. However, samples can be taken from different zones of the lesion in order to determine certain characteristics (active zone, highly squamous zone, zone displaying pronounced erythema, treated or untreated zone).

Advantageously, sampling is carried out by attaching an adhesive on the patient's skin and then removing said adhesive, to which a sample of stratum corneum adheres. The adhesive is selected such that a sufficient sample of skin is collected. The adhesive can advantageously be constituted of a flexible plastic strip, translucent or opaque. The adhesive is preferably arranged on a backing, such as a backing that is flexible and preferably pliable so that it can better adapt to the contours and to the elasticity of the part of the body on which it is applied, promoting obtaining a sample of skin, and more particularly of stratum corneum, that is as complete as possible. Said backing can be constituted for example of a plastic film. For example, the adhesive can be discs such as D-squames™ (Monaderm), Sebutape™ (CuDerm Corporation, Dallas, Tex.), Blenderm™ or ScotchTape™ (3M Company, St. Paul, Minn.), or hydrogels such as those of the Hypan™ type (Hymedix International, Inc., Dayton, N.J.), or any other kind of materials having adhesive properties such as glues, gums and resins.

According to a preferred embodiment of the method, the adhesive is applied repeatedly on the skin until it loses its adhesiveness. Alternatively it is also envisaged to use just a single application of the adhesive or to use more than one adhesive for constituting the sample to be analyzed. According to a particularly advantageous embodiment of the invention, however, it is possible to use just a single application and use only one adhesive to obtain results that can be used directly, i.e., containing sufficient biological material.

It is preferable to proceed, with the skin sample obtained in stage a), to a stage a′) of extraction of biological material. This extraction stage can be carried out according to any protocol for extraction, notably for extraction of proteins, which are well known by one skilled in the art.

The invention therefore notably features a non-invasive method for collecting biological data that can be used for establishing a diagnosis or a prognosis of a particular cutaneous pathology in a patient, as previously described, in which the sample obtained in the sampling stage a) is submitted to a stage of extraction of biological material a′), then the stage of detection b) is carried out on the extract thus obtained.

Alternatively or additionally, the skin sample can advantageously be submitted to a stage a″) of enzymatic digestion prior to analysis by mass spectrometry. This digestion stage can advantageously be carried out using trypsin. Moreover, this digestion stage can advantageously be carried out in situ directly on the backing of the adhesive, which makes it possible to speed up the analysis and reduce the amount of sample required and thus reduce the size of the sampling zone and/or make the sampling operations more precise.

This invention therefore notably features a non-invasive method for collecting biological data that can be used for establishing a diagnosis or a prognosis of a particular cutaneous pathology in a patient as previously described, in which the biological material contained in the skin sample taken in stage a) or obtained in stage a′) is submitted to a stage of enzymatic digestion a″) prior to analysis by mass spectrometry (stage b)).

To carry out the analysis and determine the presence of biological marker(s), the methods of analysis by mass spectrometry described above can be used, in particular tandem mass spectrometry MS/MS or MALDI-TOF spectrometry. According to a particular embodiment of the invention it is preferable to employ a stage of liquid chromatography, and more particularly nano-chromatography, combined with tandem mass spectrometry (MS/MS), as this method, which permits preliminary separation of complex mixtures, is particularly effective, rapid and sensitive.

Preferably, the samples are placed in a 2-ml tube (of the Eppendorf type) in the presence of a buffer solution (preferably ammonium bicarbonate pH 8.3) containing a reducing agent (preferably dithiothreitol) and a detergent compatible with analysis by mass spectrometry (preferably Rapigest™, (from the company Waters)) for dissolving the proteins, and then an alkylating agent (preferably iodoacetamide) is added (stage a′)). Digestion of the proteins with trypsin is preferably carried out directly in the tube containing the sample, at 37° C., with stirring (stage a″)). The digested sample is then filtered at 0.22 μm then 30 kDa in order to remove the cellular debris and the undigested proteins. After acidification, the peptides are loaded directly on a pre-concentration column (C18) “LC Packing” coupled to the mass spectrometer (stage b)). Elution of the pre-concentration column is preferably carried out with a continuous gradient of water:acetonitrile mixture, the peptides are separated in a column (Atlantis™) and then ionized at column outlet in the “nanospray” source for analysis in the mass spectrometer.

The mass spectra obtained are analyzed by software for interpreting mass spectra. Examples of such software are MASCOT (from the company Matrix Science), Spectrum Mill (from Agilent), Phenyx (from Genebio), and ProID (from Applied Biosystems). The software can be coupled to databases such as UNIPROT, SWISSPROT or alternatively TrEMBL.

It may be preferable to add a stage c) of separation of the peptides before the separation by nano-chromatography (stage b)) to provide better separation of the peptides resulting from enzymatic digestion. This stage is particularly recommended for complex mixtures and for the investigation of proteins present in small amounts. This preliminary stage can advantageously be constituted of a stage of (ion-exchange) liquid chromatography. The peptides from enzymatic digestion with trypsin are thus absorbed at acid pH on an (anionic) ion-exchange column and then eluted with a continuous gradient of salt of increasing concentration or with a succession of solutions containing increasing amounts of salt (“step”). The peptides thus eluted are absorbed directly (in line) on the pre-concentration column (C18) and then chromatographed as described previously.

Alternatively, the stage of preliminary separation c) can advantageously comprise a stage of isoelectric focusing of the peptides on a gel comprising a gradient of immobilin immobilized on the backing (gel). This stage is similar to the first stage of separation of the proteins carried out for two-dimensional analysis of the proteins. The application of an electric current makes it possible to separate peptides with different charges. After extraction from the gel, the peptides are then chromatographed directly as stated previously (nano-chromatography).

For these two alternatives (ion exchanger or isoelectric focusing), analysis of all of the mass spectra must be carried out in a single stage in order to identify the various peptides present in the different fractions chromatographed, and which may belong to one and the same protein.

In order to further illustrate the present invention and the advantages thereof, the following specific examples are given, it being understood that same are intended only as illustrative and in nowise limitative. In said examples to follow, all parts and percentages are given by weight, unless otherwise indicated.

### Example 1

### Proteomic Analysis of a Sample of Stratum Corneum Obtained by Application of an Adhesive Strip

a) Obtaining a Sample:

Proteomic analysis is carried out on a sample taken from the skin by applying an adhesive of the D-Squames™ type (from the company Monaderm). This product comprises a translucent flexible polymer backing and an adhesive. Application is repeated on the patient's skin, preferably until there is loss of adhesion. The sample obtained only relates to the contents of the outermost layers of the epidermis (i.e. the stratum corneum). The samples thus obtained are stored at −30° C. or at −80° C. if storage for a longer period is desired.

a′) and a″) Proteomic Analysis: Digestion:

a′) Extraction of the Biological Material:

The sample obtained is incubated for 60 min in a 2-ml Eppendorf tube, stirring continuously (at 1400 rpm) at 60° C. in 200 μl of a buffer solution of ammonium bicarbonate of pH 8.5 containing 0.1% of RapiGest™ detergent (1 mg in 1000 μl) and 5 μM of DTT (dithiothreitol). Then 15 mM of iodoacetamine (IAA) is added (6 μl of a solution at 0.5M) and it is left to incubate for 1 h30 min at room temperature in the dark.

a″) Enzymatic Digestion:

Then the sample is incubated with stirring (400 rpm) for 18 h at 37° C. with 1 μl of a solution of trypsin of porcine origin (1 mg/ml), and is then acidified with 2% formic acid (4 μl of a 10% solution). The digestion product is filtered using filters of 0.22 μm and 30 kDa with centrifugation (12,000 rpm). The filtrate is used directly for analysis by liquid chromatography coupled to mass spectrometry Qstar-XL analysis mode (LC/MS/MS).

b) Proteomic Analysis: LC/MS/MS:

It is preferable to use liquid chromatography, and more particularly nano-chromatography, combined with tandem mass spectrometry (MS/MS). The mass spectra obtained are analyzed with the MASCOT software for identification of peptides and proteins with the following specifications:

Investigation in MS/MS mode,

Type of instrument: ESI-QUAD-TOF,

Trypsin digestion,

Modification of the proteins supplied: carbamidomethyl (on the cysteines),

Monoisotope,

No tolerance on the mass of the protein, Tolerance on the peptide mass of +/−0.15 Da, Tolerance on the mass of the daughter ions (fragmentation): +/−0.1 Da,

Absence of tolerated cleavage of 1,

Search on SwissProt database: human (Homo Sapiens). This program is a search engine notably available on the Internet site www.matrixscience.com.

### Example 2

### Determination of the Presence of Markers of Psoriasis that can be Identified by Non-Invasive Sampling from Stratum Corneum

### (Identification by LC/MS/MS)

By comparison between the proteins identified that are only present in the psoriasis scales (or strongly present relative to the scales of healthy subjects) and the known biological markers of psoriasis lesions, it appears that the presence of the following markers has been identified:

| Entry number in | |
|---|---|
| SWISSPROT | Complete name |
| P31946 | protein 14-3-3 (beta/alpha) |
| P31947 | protein 14-3-3 protein (sigma) (stratifin) |
| Q9NZT1 | epidermis-specific calmodulin 5 (CLSP) |
| P62158 | Calmodulin |
| P15924 | Desmoplakin |
| Q02413 | Desmoglein 1 |
| P68104 | Elongation factor alpha 1 |
| P06733 | Alpha enolase |
| Q01469 | Protein binding the epidermal fatty acids |
| | associated with psoriasis (PA-FABP) |
| P06396 | Gelsolin |
| P08107 | Heat shock protein 70.1 (HSP71) |
| P08238 | Heat shock protein 90 |
| P04792 | Heat shock protein 27 (beta-1) |
| P07476 | Involucrin |
| P02533 | Keratin 14 |
| P08779 | Keratin 16 |
| Q04695 | Keratin 17 |
| P02538 | Keratin, type II cytoskeletal 6A |
| P04259 | Keratin, type II cytoskeletal 6B |
| P47929 | Galectin 7 |
| P29034 | Protein S100 A2 |
| P31151 | Psoriasin (S100 A7) |
| P05109 | calgranulin A (S100A8) |
| P06702 | calgranulin B (S100A9) |
| P29508 | Squamous cell carcinoma antigen 1 (SCCA1) |
| P48594 | Squamous cell carcinoma antigen 2 (SCCA2) |
| Q08188 | Transglutaminase 3 (Tgase E) |
| P10599 | Thioredoxin |
| P36952 | Maspin |
| P19971 | Thymidine phosphorylase |

Said markers can be used advantageously in a method according to the invention.

Each patent, patent application, publication, text and literature article/report cited or indicated herein is hereby expressly incorporated by reference in its entirety.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A non-invasive method for developing biological data useful for establishing a diagnosis or a prognosis of a particular cutaneous pathology in a patient, comprising:
a) obtaining a skin sample containing biological material by applying a layer of adhesive on the skin of such patient, and then removing said adhesive containing such skin sample/biological material, and
b) conducting mass spectrometry and detecting, in the skin sample obtained in a), at least one protein whose presence, absence, or variation in amount or concentration relative to a standard value is associated with the presence, the development or the absence of a particular cutaneous pathology.

2. The non-invasive method as defined by claim 1, wherein the cutaneous pathology is psoriasis.

3. The non-invasive method as defined by claim 1, wherein the skin sample obtained in a) is submitted to a stage a′) of extraction of biological material, then the stage of detection b) is carried out on the extract thus obtained.

4. The non-invasive method as defined by claim 1, wherein the biological material contained in the skin sample obtained in stage a) or extracted in a stage a′) is subjected to a stage a″) of enzymatic digestion prior to stage b).

5. The non-invasive method as defined by claim 1, wherein the technique of mass spectrometry conducted is tandem mass spectrometry (MS/MS).

6. The non-invasive method as defined by claim 5, wherein the analysis by mass spectrometry comprises a stage of separation by liquid chromatography, combined with tandem mass spectrometry (MS/MS).

7. The non-invasive method as defined by claim 1, wherein said at least one protein is a marker of psoriatic skin lesions.

8. The non-invasive method as defined by claim 7, wherein said marker is selected from the group comprising proteins having biological functions associated with cellular proliferation and differentiation.

9. The non-invasive method as defined by claim 8, wherein said marker is selected from the group consisting of stratifin, calmodulin-like epidermis-specific protein 5, protein binding fatty acids associated with psoriasis, desmoplakin, psoriasin, alpha enolase, and squamous cell carcinoma antigens 1 and 2.

10. The non-invasive method as defined by claim 7, wherein said marker is selected from the group consisting of proteins having biological functions connected with the response to stress and with the immune and inflammatory response.

11. The non-invasive method as defined by claim 10, wherein said marker is selected from the group consisting of the heat shock proteins 27 (beta 1), 70.1, and 90, and Calgranulin A and B.

12. The non-invasive method as defined by claim 1, comprising obtaining a single sample.
